(19) United States
(12) Patent Application Publication
(10) Pub. No.: US 2020/0303034 A1
(43) Pub. Date: Sep. 24, 2020

(54) METHODS, SYSTEMS, APPARATUSES AND DEVICES FOR ACCELERATING EXECUTION OF A SEARCH QUERY FOR PEPTIDE IDENTIFICATION

(71) Applicant: INTEGRATED PROTEOMICS APPLICATIONS INC., San Diego, CA (US)

(72) Inventors: Robin Park, San Diego, CA (US); Titus Jung, San Diego, CA (US)

(73) Assignee: INTEGRATED PROTEOMICS APPLICATIONS INC., San Diego, CA (US)

(21) Appl. No.: 16/357,296

(22) Filed: Mar. 18, 2019

(51) Int. Cl. G16B 20/00

(52) U.S. Cl. CPC G16B 20/00

(57) ABSTRACT

A system for accelerating execution of a search query for peptide identification is disclosed. The system may include a communication device configured for receiving a spectral file including mass spectrometry-based proteomics data from a user device. Further, the system may include a processing device configured for splitting the spectral file into spectral split files based on precursor mass, identifying candidate peptides based on querying, combining protein identification scores, and identifying a peptide corresponding to the mass spectrometry-based proteomics data based on the combining. Further, the system may include a protein database configured for querying based on the plurality of spectral split files. Further, the system may include a plurality of GPU cores communicatively coupled to the processing device configured for computing the plurality of protein identification scores corresponding to candidate peptides.

### TECHNICAL FIELD

Generally, the present disclosure relates to the field of data processing. More specifically, the present disclosure relates to methods, systems, apparatuses and devices for accelerating execution of a search query for peptide identification.

### BACKGROUND

The field of proteomics is technologically important to several industries, business organizations and/or individuals. In particular, the use of proteomics, and peptide study and identification is prevalent for deciphering how proteins interact as a system and for comprehending the functions of cellular systems in human disease. The progress of techniques related to proteomics has permitted an in-depth investigation of molecular mechanisms underlying in diseases, such as cardiovascular diseases. Accordingly, advance in proteomics techniques has also enabled the identification of proteins, and the nature of the associated modification. Further, proteomics is becoming a part of the quality-control process in transfusion medicine with verification of identity, safety, potency and purity of various blood products being an object of study.

Existing techniques for peptide identification are deficient with regard to several aspects. For instance, current technologies for searching for peptides tend to be slower.

Furthermore, current software may not be optimized for searching for protein databases rapidly through parallel computing

Therefore, there is a need for improved methods, systems, apparatuses and devices for accelerating execution of peptide identification that may overcome one or more of the above-mentioned problems and/or limitations.

### BRIEF SUMMARY

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

Disclosed herein is a method of accelerating execution of a search query for peptide identification, in accordance with some embodiments. Accordingly, the method may include a step of receiving, using a communication device, a spectral file including mass spectrometry-based proteomics data from a user device. Further, the method may include a step of splitting, using a processing device, the spectral file into spectral split files based on precursor mass. Further, the method may include a step of querying, using a protein database, based on the plurality of spectral split files. Further, the method may include a step of identifying, using the processing device, candidate peptides based on the querying. Further, the method may include a step of computing, using GPU cores, protein identification scores corresponding to candidate peptides. Further, the method may include a step of combining, using the processing device, the plurality of protein identification scores. Further, the method may include a step of identifying, using the processing device, a peptide corresponding to the mass spectrometry-based proteomics data based on the combining.

Further disclosed herein is a system for accelerating execution of a search query for peptide identification, in accordance with some embodiments. Accordingly, the system may include a communication device configured for receiving a spectral file including mass spectrometry-based proteomics data from a user device. Further, the system may include a processing device configured for splitting the spectral file into spectral split files based on precursor mass. Further, the processing device may be configured for identifying candidate peptides based on querying. Further, the processing device may be configured for combining protein identification scores. Further, the processing device may be configured for identifying a peptide corresponding to the mass spectrometry-based proteomics data based on the combining. Further, the system may include a protein database configured for querying based on the plurality of spectral split files. Further, the system may include GPU cores communicatively coupled to the processing device configured for computing the plurality of protein identification scores corresponding to candidate peptides

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 is an illustration of an online platform consistent with various embodiments of the present disclosure.

FIG. 2 is a system of accelerating execution of a search query for peptide identification, in accordance with some embodiments.

FIG. 3 is a flowchart of a method of accelerating execution of a search query for peptide identification, in accordance with some embodiments.

FIG. 4 is a flowchart of a method of launching virtual machine instances based on computational time, in accordance with some embodiments.

FIG. 5 is a flowchart of a method of identification of a protein using Graphics Processing Units (GPUs), in accordance with some embodiments.

FIG. 6 is an exemplary architecture of a system of accelerating execution of a search query for peptide identification, in accordance with some embodiments.

FIG. 7 is an exemplary architecture of a system of accelerating execution of a search query for peptide identification, including GPU cores, in accordance with some embodiments.

FIG. 8 is a graph showing GPU search speed in comparison with CPU search speed related to the execution of a search query for peptide identification, in accordance with some embodiments.

FIG. 9 shows an integrated proteomics pipeline in communication with a GPU cluster including GPUs, in accordance with some embodiments.

FIG. 10 is a block diagram of a computing device for implementing the methods disclosed herein, in accordance with some embodiments.

### DETAILED DESCRIPTION

As a preliminary matter, it will readily be understood by one having ordinary skill in the relevant art that the present disclosure has broad utility and application. As should be understood, any embodiment may incorporate only one or the above-disclosed aspects of the disclosure and may further incorporate only one or a plurality of the above-disclosed features. Furthermore, any embodiment discussed and identified as being “preferred” is considered to be part of the best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim limitation found herein and/or issuing here from that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the claims found herein and/or issuing here from. The present disclosure contains headers. It should be understood that these headers are used as references and are not to be construed as limiting upon the subject matter disclosed under the header.

The present disclosure includes many aspects and features. Moreover, while many aspects and features relate to, and are described in the context of accelerating execution of a search query for peptide identification, embodiments of the present disclosure are not limited to use only in this context.

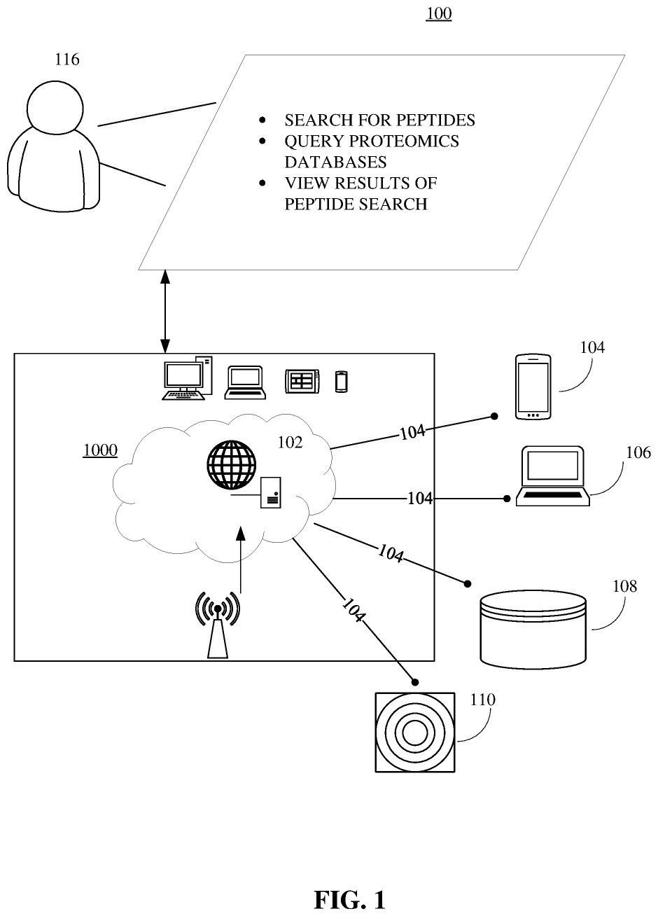

FIG. 1 is an illustration of an online platform 100 consistent with various embodiments of the present disclosure. By way of non-limiting example, the online platform 100 to facilitate accelerating execution of a search query for peptide identification may be hosted on a centralized server 102, such as, for example, a cloud computing service. The centralized server 102 may communicate with other network entities, such as, for example, a mobile device 104 (such as a smartphone, a laptop, a tablet computer etc.), other electronic devices 106 (such as desktop computers, server computers etc.), databases 108, and sensors 110 over a communication network 114, such as, but not limited to, the Internet. Further, users of the online platform 100 may include relevant parties such as, but not limited to, end users, administrators, service providers, service consumers and so on. Accordingly, in some instances, electronic devices operated by one or more relevant parties may be in communication with the platform.

A user 116, such as the one or more relevant parties, may access online platform 100 through a web-based software application or browser. The web-based software application may be embodied as, for example, but not be limited to, a website, a web application, a desktop application, and a mobile application compatible with a computing device 1000.

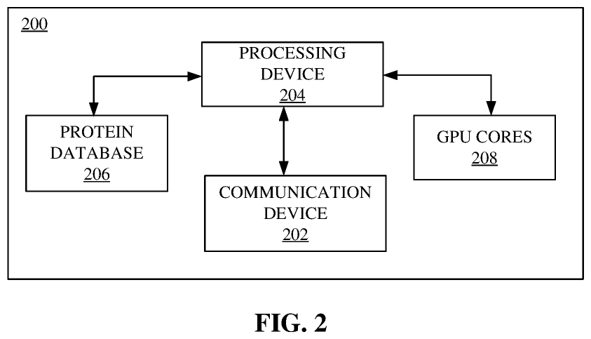

FIG. 2 is a system 200 of accelerating execution of a search query for peptide identification, in accordance with some embodiments. Further, the system 200 may include a communication device 202 configured for receiving a spectral file including mass spectrometry-based proteomics data from a user device. Further, the system 200 may include a processing device 204 communicatively coupled to the communication device 202. Further, the processing device 204 may be configured for splitting the spectral file into spectral split files based on precursor mass. Further, each spectral split file may include mass spectrometry-based proteomics data corresponding to a predetermined range of precursor masses. Further, a smaller spectral file of the plurality of spectral split files may only contain spectra between a given range of precursor masses, allowing for a decrease in query time and memory usage when querying for peptide candidates. Further, the processing device 204 may be configured for identifying candidate peptides based on querying. Further, the processing device 204 may be configured for combining protein identification scores. Further, the processing device 204 may be configured for identifying a peptide corresponding to the mass spectrometry-based proteomics data based on the combining. Further, the system 200 may include a protein database 206 configured for querying based on the plurality of spectral split files. Further, in an embodiment, the protein database 206 may include an SQLite database, such as a protein database 708. Further, the system 200 may include GPU cores 208 communicatively coupled to the processing device 204. Further, the plurality of GPU cores 208 may be configured for computing the plurality of protein identification scores corresponding to candidate peptides. Further, the computing may be performed in parallel across the plurality of GPU cores 208.

In some embodiments, the search query may correspond to a Post-Translational Modification (PTM) search.

In some embodiments, the plurality of protein identification scores may include preliminary PSM (peptide-spectrum match) scores. Further, the plurality of preliminary PSM scores may be calculated through a scoring function available in GPU cores operating in parallel. Further, a job scheduler may manage a large number of spectra to be processed in a CPU-GPU search pipeline.

In some embodiments, the processing device 204 may be further configured for identifying the top-N number of candidate peptides from the plurality of candidate peptides based on the plurality of protein identification scores. Further, the combining of the plurality of protein identification scores may correspond to the top-N number of candidate peptides. Further, in an embodiment, the combining of the plurality of protein identification scores may lead to a generation of a final main score. Further, the final main score may be generated by running a highly optimized matrix multiplication algorithm with theoretical peaks on the plurality of split spectral files. Further, the plurality of protein identification scores may be retrieved from the plurality of GPU cores, may be used to generate the final main score.

In some embodiments, the plurality of GPU cores 208 may be comprised in a cluster of GPU cards including modular GPU cards. Further, each modular GPU card may include two or more GPU cores 208. Further, in an embodiment, a number of the plurality of modular GPU cards may be increased in the cluster of GPU cards.

In some embodiments, the system 200 may further include a memory device configured for storing indicators of the plurality of candidate peptides using primitive data arrays.

In some embodiments, the processing device 204 may include at least one CPU core.

In some embodiments, the processing device 204 may be further configured for determining a computational time based on the analyzing. Further, the computation time may include an estimated time duration for performing the peptide identification. Further, the processing device 204 may be configured for launching virtual machine instances based on the computational time.

In some embodiments, a speed of execution of the search query using the plurality of GPU cores 208 may be roughly 100 times faster than a corresponding speed of execution of the search query using a CPU core. Further, in some embodiments, the speed of execution of the search query using the plurality of GPU cores 208 may be increased by increasing the number of the plurality of GPU cores 208.

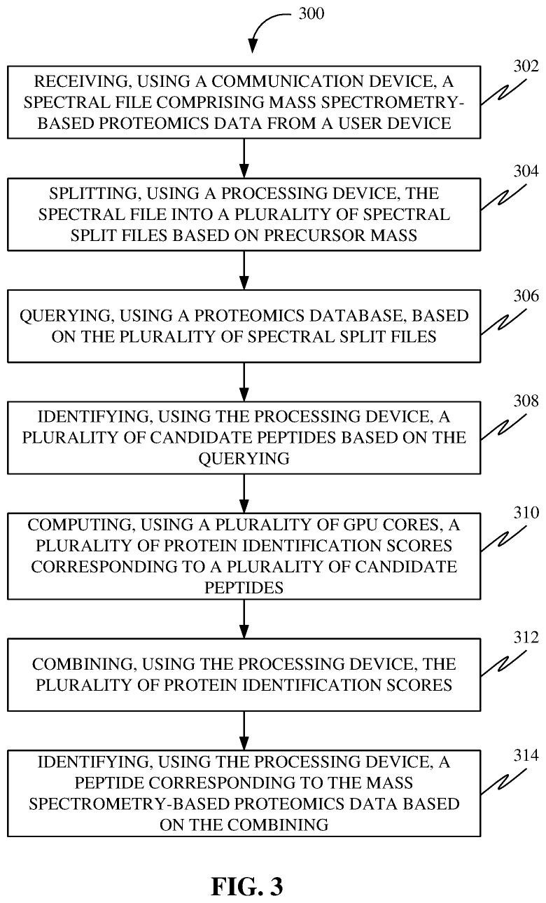

FIG. 3 is a flowchart of a method 300 of accelerating execution of a search query for peptide identification, in accordance with some embodiments. Further, at 302, the method 300 may include receiving, using a communication device, such as the communication device 202, a spectral file including mass spectrometry-based proteomics data from a user device.

Further, at 304, the method 300 may include splitting, using a processing device, such as the processing device 204, the spectral file into spectral split files based on precursor mass. Further, each spectral split file may include mass spectrometry-based proteomics data corresponding to a predetermined range of precursor masses.

Further, at 306, the method 300 may include querying, using a protein database (such as the protein database 206), based on the plurality of spectral split files.

Further, at 308, the method 300 may include identifying, using the processing device, candidate peptides based on the querying.

Further, at 310, the method 300 may include computing, using GPU cores, such as the GPU cores 208, protein identification scores corresponding to candidate peptides. Further, the computing may be performed in parallel across the plurality of GPU cores.

Further, at 312, the method 300 may include combining, using the processing device, the plurality of protein identification scores.

Further, at 314, the method 300 may include identifying, using the processing device, a peptide corresponding to the mass spectrometry-based proteomics data based on the combining.

In some embodiments, the search query may correspond to a Post-Translational Modification (PTM) search.

In some embodiments, the plurality of protein identification scores may include preliminary PSM (peptide-spectrum match) scores.

In some embodiments, method 300 may further include identifying, using the processing device, a top-N number of candidate peptides from the plurality of candidate peptides based on the plurality of protein identification scores. Further, the combining of the plurality of protein identification scores may correspond to the top-N number of candidate peptides.

In some embodiments, the plurality of GPU cores may be comprised in a cluster of GPU cards including modular GPU cards. Further, each modular GPU card may include two or more GPU cores.

In some embodiments, method 300 may further include storing, using a memory device, indicators of the plurality of candidate peptides using primitive data arrays.

In some embodiments, the processing device may include at least one CPU core.

In some embodiments, the search space may include all fully-tryptic and half-tryptic peptide candidates falling within a mass tolerance window with no miscleavage constraints.

In some embodiments, a speed of execution of the search query using the plurality of GPU cores may be at least 80 times faster than a corresponding speed of execution of the search query using a CPU core.

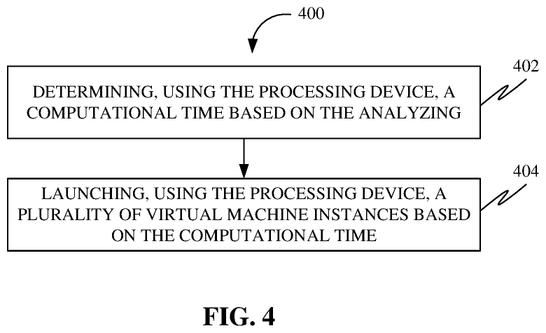

FIG. 4 is a flowchart of a method 400 of launching virtual machine instances based on computational time, in accordance with some embodiments. Further, at 402, the method 400 may include determining, using the processing device, a computational time based on the analyzing. Further, the computation time may include an estimated time duration for performing the peptide identification.

Further, at 404, the method 400 may include launching, using the processing device, virtual machine instances based on the computational time.

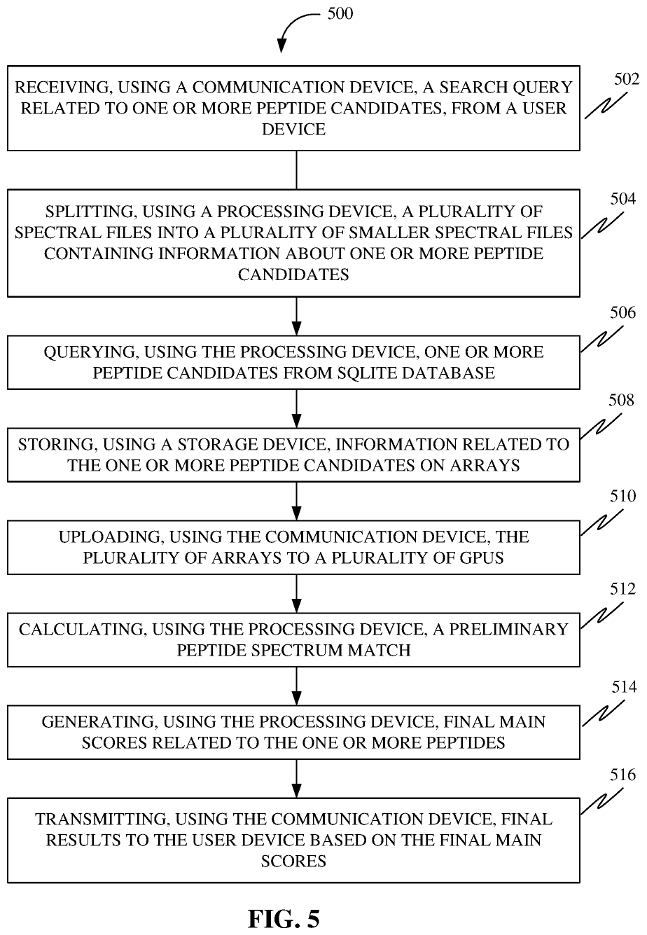

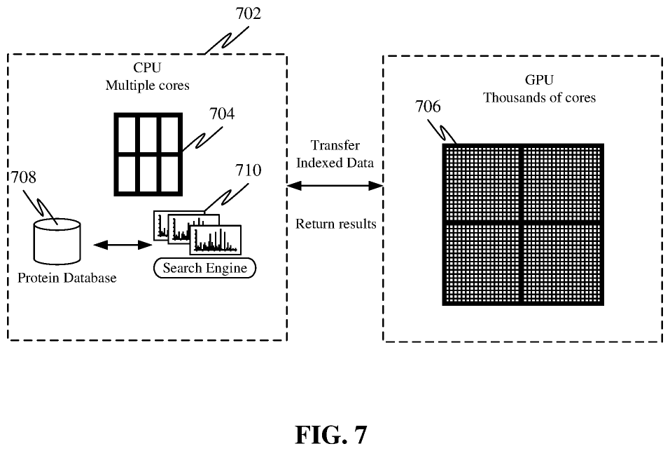

FIG. 5 is a flowchart of a method 500 of identification of a protein using Graphics Processing Units (GPUs), in accordance with some embodiments. Further, at 502, the method 500 may include receiving, using a communication device, a search query related to one or more peptide candidates, from a user device. Further, the search query may be related to protein identification in proteomics experiments. Further, in an instance, the search query may be received through an input mechanism of the user device. For instance, the user device may include one or more of a smartphone, a laptop computer, a desktop computer, a tablet computer, and so on. Accordingly, as shown in FIG. 7, the search query may be received from the user device through a search engine 710.

Further, at 504, the method 500 may include splitting, using a processing device, spectral files into smaller spectral files containing information about one or more peptide candidates. Further, the plurality of spectral files may store spectroscopic data, such as related to tandem mass spectrometry. Further, the plurality of spectral files may be split into smaller files by precursor mass. Further, precursor mass may describe the mass of ions that may have dissociated into smaller fragment ions, such as due to collision-induced dissociation in a multistage/mass spectrometry experiment, such as tandem mass spectrometry. Further, a smaller spectral file of the plurality of smaller spectral files may only contain spectra between a given range of precursor masses, allowing for a decrease in query time and memory usage when querying for peptide candidates. Further, the decrease in the query time and the memory usage may result from looking for candidate peptides within a particular search range corresponding to the search query. Further, when used in a cluster environment, the splitting of the plurality of spectral files may improve memory efficiency and performance, since each search job may only load peptide candidates within a mass range.

Further, at 506, the method 500 may include querying, using the processing device, one or more peptide candidates from an SQLite database. For instance, as shown in FIG. 7, the one or more peptide candidates may be queried from the protein database 708 (SQLite database).

Further, at 508, the method 500 may include storing, using a storage device, information related to the one or more peptide candidates on arrays. Further, information related to the one or more peptide candidates may be stored on primitive data arrays. Further, in an embodiment, information related to the one or more peptide candidates may be stored on non-primitive data arrays. Further, the storing of the information on arrays may reduce memory usage on a CPU side compared to storing information on objects, and may allow for easy transfer to GPU memory associated with the plurality of GPUs.

Further, at 510, the method 500 may include uploading, using the communication device, the plurality of arrays to GPUs. Further, the plurality of arrays may be processed and uploaded from the CPU side to the plurality of GPUs in large batches. For instance, as shown in FIG. 7, the plurality of arrays may be uploaded from CPU side 702, including a CPU 704 with one or more cores, to GPUs 706 with a plurality of cores. Further, the processing and uploading of the plurality of arrays in large batches may lead to a reduced GPU upload time.

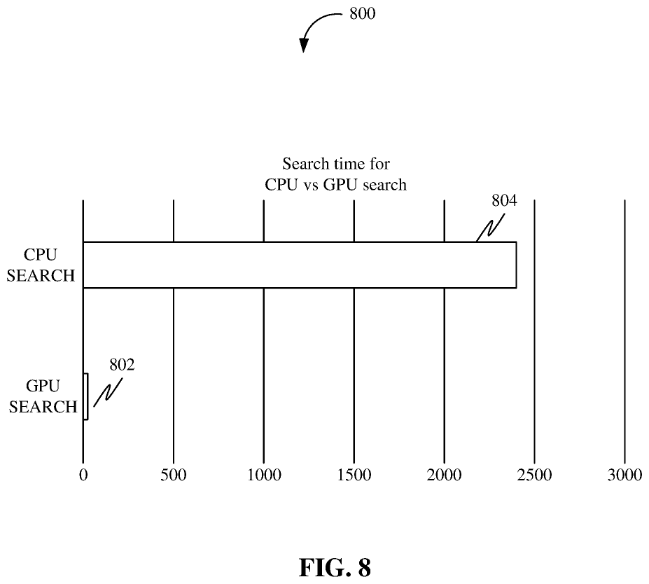

Further, at 512, the method 500 may include calculating, using the processing device, a preliminary peptide-spectrum match. Further, the preliminary PSM (peptide-spectrum match) scores may be calculated for a large number of peptide candidates in parallel using GPU cores. Further, while calculating the preliminary PSM scores, only a top N number of peptide candidates may be taken into consideration. Further, other peptide candidates may be rapidly discarded. For instance, if top 10 peptide candidates are taken into consideration based on the preliminary PSM scores, the rest of the peptide candidates may be discarded. Further, the top 10 peptide candidates may change and may be discarded based on the preliminary PSM scores. Further, taking only a top N number of peptide candidates into consideration may reduce memory usage as only a small fraction of peptide candidates may be kept before calculating a final main score. Further, the preliminary PSM scores may be calculated through a scoring function in NVIDIA® GPU cards in parallel, such as in a CUDA® module. Further, a job scheduler may manage a large number of spectra to be processed in a CPU-GPU search pipeline. Further, an unlimited GPU computer clusters may be used to run the scoring function, leading to an increased search speed. For instance, as shown in FIG. 8 with graph 800, GPU search speed 802 is approximately 80 times faster than CPU search speed 804.

Further, at 514, the method 500 may include generating, using the processing device, the final main score related to the one or more peptides. Further, the final main score may be generated by running a highly optimized matrix multiplication algorithm with theoretical peaks and spectral data. The preliminary PSM scores may be retrieved from the plurality of GPUs and may be used to generate the final main score. Further, the preliminary PSM scores may be returned to the CPU side (CPU search module) to combine and generate the final main score.

Further, at 516, the method 500 may include transmitting, using the communication device, the final results to the user device based on the final main score.

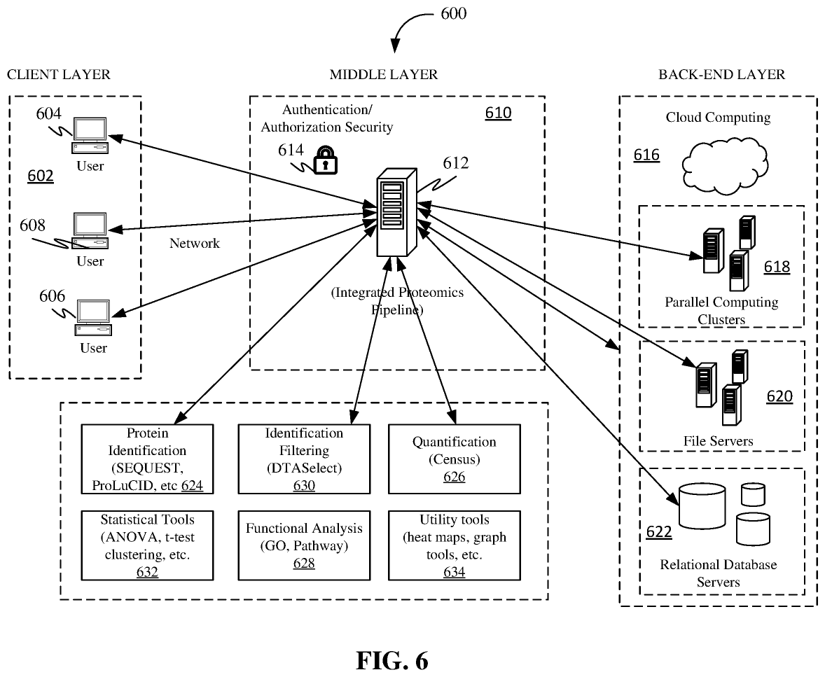

FIG. 6 is an exemplary architecture 600 of a system of accelerating execution of a search query for peptide identification, in accordance with some embodiments. Further, the architecture 600 may include a client layer 602 including a plurality of users (such as user 604, user 606, and user 608). Further, the plurality of users may access the system from a plurality of user devices, such as the mobile device 104 (including smartphones), the electronic devices 106 (including laptop computers, desktop computers, tablet computers), and so on. Further, the plurality of users may access the system through a web-based software application or browser. Further, the web-based software application may be embodied as, for example, but not be limited to, a website, a web application, a desktop application, and a mobile application compatible with the plurality of user devices associated with the plurality of users.

Further, the architecture 600 may include a middle layer 610, including an integrated proteomics pipeline 612. Further, the integrated proteomics pipeline 612 may make use of MySQL® or Oracle® database to store proteomics metadata. Further, in an embodiment, the integrated proteomics pipeline 612 may make use of MongoDB® database to accommodate extremely large protein database (e.g. microbiome databases bigger than 40 gigabytes) for fast search. Further, in an embodiment, the integrated proteomics pipeline 612 may run on the cloud. Further, a user, such as the user 606 of the plurality of users may submit a data analysis job, the integrated proteomics pipeline 612 may automatically calculate an amount of computational time and may launch a number of EC2 instances from customized AMI through a secured connection. Further, a cloud module of integrated proteomics pipeline 612 may support Amazon Web Services® (AWS) and Microsoft Azure® clouds to perform high-throughput proteomics data. Further, in an embodiment, the integrated proteomics pipeline 612 may be implemented as a local computational clustering infrastructure, allowing cluster modules associated with the integrated proteomics pipeline 612 to perform the analysis. Further, the integrated proteomics pipeline 612 may include an authorization and security module 614 to facilitate authorization of the plurality of users.

Further, the architecture 600 may include a back end layer 616, including parallel computing clusters 618, file servers 620, and relational database servers 622.

Further, a plurality of data analysis software may be integrated into the integrated proteomics pipeline 612 providing a single and consistent user interface to allow a user of the plurality of users, such as the user 608 to process big biomedical data in an easy way. Further, the integrated proteomics pipeline 612 may include a highly sensitive protein identification software 624 providing protein identification results, such as with ProLuCID® search engine. Further, the integrated proteomics pipeline 612 may include a quantitative analysis software 626, supporting quantitative analyses including 15N metabolic labeling, Stable Isotope Labeling by Amino Acids In Cell culture (SILAC), Isobaric Tag For Relative And Absolute Quantitation (iTRAQ), Tandem Mass Tag (TMT) and label-free by using Census®, a comprehensive quantitative analysis tool. A user, such as the user 604, such as a researcher may organize experiments with project organization tools and compare a large number of samples quickly and confidently to identify proteins/peptides of interest. Further, the integrated proteomics pipeline 612 may include a functional analysis software 628 allowing for functional analysis with methods such as GO, and pathway, identification filtering software 630 including tools such as DTAselect®, statistical tools 632, such as analysis of variance (ANOVA), t-test clustering, and so on, and utility tools 634, including heat maps, graph tools, and so on. Further, the integrated proteomics pipeline 612 may allow running of third-party software through an Application Programming Interface of the integrated proteomics pipeline 612 (known as for e.g. IP2-API). Further, projects initiated on the integrated proteomics pipeline 612 may be shared amongst the plurality of users and may be published to public repositories.

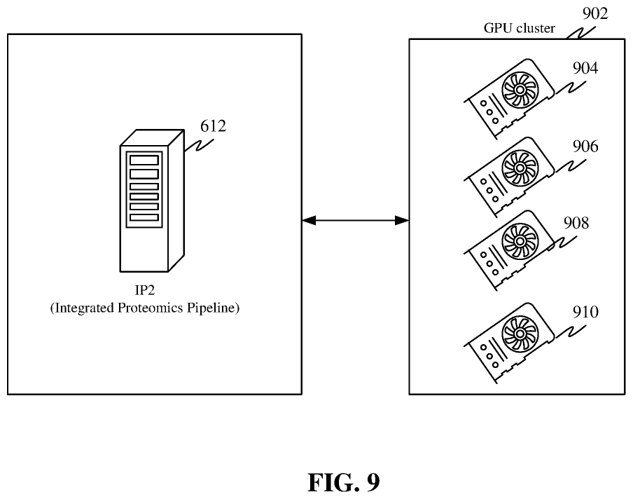

Further, in an embodiment, as shown in FIG. 9, the integrated proteomics pipeline 612 may be connected to a GPU cluster 902 including a plurality of GPUs, including a first GPU 904, a second GPU 906, a third GPU 908, and a fourth GPU 910.

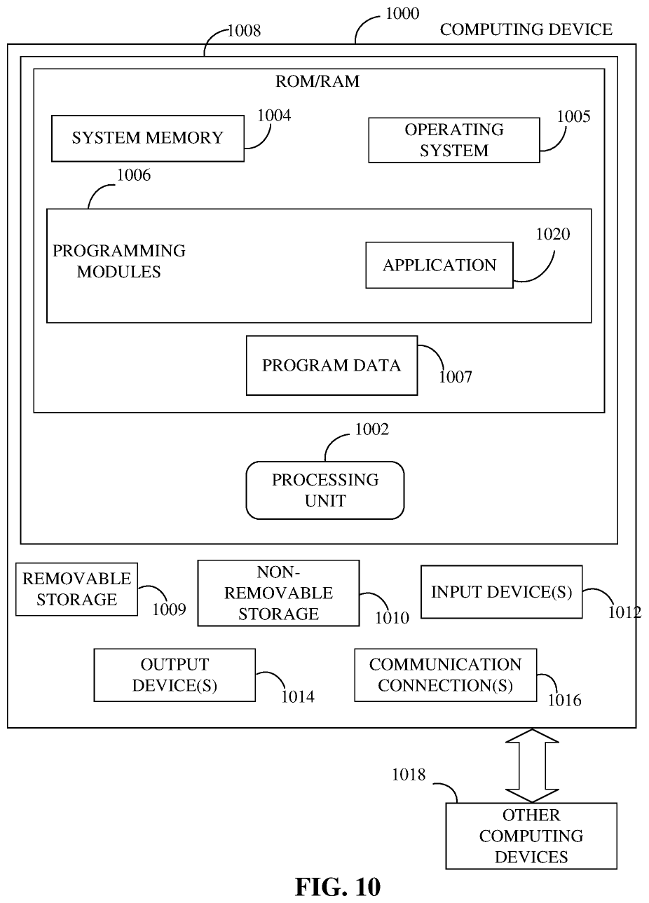

With reference to FIG. 10, a system consistent with an embodiment of the disclosure may include a computing device or cloud service, such as computing device 1000. In a basic configuration, computing device 1000 may include at least one processing unit 1002 and system memory 1004. Depending on the configuration and type of computing device, a system memory 1004 may comprise, but is not limited to, volatile (e.g. random-access memory (RAM)), non-volatile (e.g. read-only memory (ROM)), flash memory, or any combination. System memory 1004 may include operating system 1005, one or more programming modules 1006, and may include a program data 1007. Operating system 1005, for example, may be suitable for controlling computing device 1000′s operation. In one embodiment, programming modules 1006 may include a machine learning module. Furthermore, embodiments of the disclosure may be practiced in conjunction with a graphics library, other operating systems, or any other application program and are not limited to any particular application or system. This basic configuration is illustrated in FIG. 10 by those components within a dashed line 1008.

Computing device 1000 may have additional features or functionality. For example, a computing device 1000 may also include additional data storage devices (removable and/or non-removable) such as, for example, magnetic disks, optical disks, or tape. Such additional storage is illustrated in FIG. 10 by a removable storage 1009 and a non-removable storage 1010. Computer storage media may include volatile and nonvolatile, removable and non-removable media implemented in any method or technology for storage of information, such as computer-readable instructions, data structures, program modules, or other data. System memory 1004, removable storage 1009, and non-removable storage 1010 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 1000. Any such computer storage media may be part of device 1000. Computing device 1000 may also have input device(s) 1012 such as a keyboard, a mouse, a pen, a sound input device, a touch input device, a location sensor, a camera, a biometric sensor, etc. Output device(s) 1014 such as a display, speakers, a printer, etc. may also be included. The aforementioned devices are examples and others may be used.

Computing device 1000 may also contain a communication connection 1016 that may allow device 1000 to communicate with other computing devices 1018, such as over a network in a distributed computing environment, for example, an intranet or the Internet. Communication connection 1016 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer-readable media as used herein may include both storage media and communication media.

As stated above, a number of program modules and data files may be stored in system memory 1004, including operating system 1005. While executing on processing unit 1002, programming modules 1006 (e.g., application 1020 such as a media player) may perform processes including, for example, one or more stages of methods, algorithms, systems, applications, servers, databases as described above. The aforementioned process is an example, and processing unit 1002 may perform other processes. Other programming modules that may be used in accordance with embodiments of the present disclosure may include a machine learning application.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general purpose graphics processor-based systems, multiprocessor systems, microprocessor-based or programmable consumer electronics, application specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufactures, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example, but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer usable or computer-readable medium could even be paper or another suitable medium upon which the program is printed, as the program can be electronically captured, via, for instance, optical scanning of the paper or another medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may, in fact, be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid-state storage (e.g., USB drive), or a CD-ROM, a carrier wave from the Internet, or other forms of RAM or ROM.

Further, the disclosed methods' stages may be modified in any manner, including by reordering stages and/or inserting or deleting stages, without departing from the disclosure.

Although the disclosure has been explained in relation to its preferred embodiment, it is to be understood that many other possible modifications and variations can be made without departing from the spirit and scope of the disclosure.

What is claimed is:

1. A method of accelerating execution of a search query for peptide identification, the method comprising:
receiving, using a communication device, a spectral file comprising mass spectrometry-based proteomics data from a user device;
splitting, using a processing device, the spectral file into spectral split files based on precursor mass, wherein each spectral split file comprises mass spectrometry-based proteomics data corresponding to a predetermined range of precursor masses;
querying, using a protein database, based on the plurality of spectral split files;
identifying, using the processing device, candidate peptides based on the querying;
computing, using a plurality of GPU cores, protein identification scores corresponding to candidate peptides, wherein the computing is performed in parallel across the plurality of GPU cores;
combining, using the processing device, the plurality of protein identification scores; and
identifying, using the processing device, a peptide corresponding to the mass spectrometry-based proteomics data based on the combining.

2. The method of claim 1, wherein the search query corresponds to a Post-Translational Modification (PTM) search.

3. The method of claim 1, wherein the plurality of protein identification scores comprises preliminary PSM (peptide-spectrum match) scores.

4. The method of claim 1 further comprising identifying, using the processing device, a top-N number of candidate peptides from the plurality of candidate peptides based on the plurality of protein identification scores, wherein the combining of the plurality of protein identification scores corresponds to the top-N number of candidate peptides.

5. The method of claim 1, wherein the plurality of GPU cores is comprised in a cluster of GPU cards comprising a plurality of modular GPU cards, wherein each modular GPU card comprises two or more GPU cores.

6. The method of claim 1 further comprising storing, using a memory device, indicators of the plurality of candidate peptides using primitive data arrays.

7. The method of claim 1, wherein the processing device comprises at least one CPU core.

8. The method of claim 1, wherein the search space comprises all fully-tryptic and half-tryptic peptide candidates falling within a mass tolerance window with no miscleavage constraints.

9. The method of claim 1 further comprising:
determining, using the processing device, a computational time based on the analyzing, wherein the computation time comprises an estimated time duration for performing the peptide identification; and
launching, using the processing device, a plurality of virtual machine instances based on the computational time.

10. The method of claim 1, wherein a speed of execution of the search query using the plurality of GPU cores is at least 100 times faster than a corresponding speed of execution of the search query using a CPU core.

11. A system of accelerating execution of a search query for peptide identification, the system comprising:
a communication device configured for receiving a spectral file comprising mass spectrometry-based proteomics data from a user device;
a processing device communicatively coupled to the communication device, wherein the processing device is configured for:
splitting the spectral file into spectral split files based on precursor mass, wherein each spectral split file comprises mass spectrometry-based proteomics data corresponding to a predetermined range of precursor masses;
identifying candidate peptides based on querying;
combining protein identification scores; and
identifying a peptide corresponding to the mass spectrometry-based proteomics data based on the combining;
a protein database configured for querying based on the plurality of spectral split files; and
a plurality of GPU cores communicatively coupled to the processing device, wherein the plurality of GPU cores is configured for computing the plurality of protein identification scores corresponding to candidate peptides, wherein the computing is performed in parallel across the plurality of GPU cores;

12. The system of claim 11, wherein the search query corresponds to a Post-Translational Modification (PTM) search.

13. The system of claim 11, wherein the plurality of protein identification scores comprises preliminary PSM (peptide-spectrum match) scores.

14. The system of claim 1, wherein the processing device is further configured for identifying a top-N number of candidate peptides from the plurality of candidate peptides based on the plurality of protein identification scores, wherein the combining of the plurality of protein identification scores corresponds to the top-N number of candidate peptides.

15. The system of claim 11, wherein the plurality of GPU cores is comprised in a cluster of GPU cards comprising a plurality of modular GPU cards, wherein each modular GPU card comprises two or more GPU cores.

16. The system of claim 11 further comprising a memory device configured for storing indicators of the plurality of candidate peptides using primitive data arrays.

17. The system of claim 11, wherein the processing device comprises at least one CPU core.

18. The system of claim 11, wherein the search space comprises all fully-tryptic and half-tryptic peptide candidates falling within a mass tolerance window with no miscleavage constraints.

19. The system of claim 11, wherein the processing device is further configured for:
determining a computational time based on the analyzing, wherein the computation time comprises an estimated time duration for performing the peptide identification; and
launching a plurality of virtual machine instances based on the computational time.

20. The system of claim 11, wherein a speed of execution of the search query using the plurality of GPU cores is at least 100 times faster than a corresponding speed of execution of the search query using a CPU core.
